(19) United States
(12) Patent Application Publication
(10) Pub. No.: US 2013/0068238 A1
(43) Pub. Date: Mar. 21, 2013

(54) Ear Protector and Ear Protector Wrap

(71) Applicants: Marcia Helena Parris, Cookeville, TN (US); Ricky Curtis Sherrell, Cookeville, TN (US)

(72) Inventors: Marcia Helena Parris, Cookeville, TN (US); Ricky Curtis Sherrell, Cookeville, TN (US)

(21) Appl. No.: 13/674,706

(22) Filed: Nov. 12, 2012

Related U.S. Application Data
(63) Continuation-in-part of application No. 13/183,032, filed on Jul. 14, 2011.
(60) Provisional application No. 61/367,716, filed on Jul. 26, 2010.

(51) Int. Cl. A61F 11/14

(52) U.S. Cl. 128/866

(57) ABSTRACT

The present disclosure provides an ear protector and an ear protector wrap. In some embodiments, the ear protector comprises: an inner wall, an outer wall, and a curved joining wall. The ear protector is manufactured with a material this is flexible, able to resume its original shape when a deforming force is removed, light weight and low density, has a high compression set, is has insulation properties and chemical resistance properties, easy to manufacture easily colored and processed. The ear protector may include a notch with thin membrane for stretch and retraction, ear cavity button for securing with ear, and thicker bridges for strength and comfort, and installing and removing ear protector with ear. In other embodiments, the present disclosure provides an ear protector wrap for attaching medical tubing to a helix of a human ear.

### CROSS-REFERENCE

The present application is a Continuation-In-Part of the parent U.S. patent application Ser. No. 13/183,032, filed Jul. 14, 2011, titled Ear Protector, currently pending which claims the benefit of U.S. Provisional Application Ser. No. 61/367,716, filed Jul. 26, 2010, titled EAR PROTECTOR. The present application is related by the same inventors for all applications, Marcia Helena Parris and Ricky Curtis Sherrell. The parent application U.S. patent application Ser. No. 13/183,032, filed Jul. 14, 2011 is hereby incorporated in its entirety by reference. In addition, U.S. Provisional Application Ser. No. 61/367,716, filed Jul. 26, 2010, titled EAR PROTECTOR is hereby incorporated in its entirety by reference.

### FIELD OF INVENTION

The present invention relates to ear protection for the human ear. More specifically, an ear protector providing a means for attaching with the ear and providing protection from heat, chemicals, noise, and positioning the ear for hair treatments as well as irritation from medical equipment such as oxygen tubing that comes in contact with the ear. An ear protector wrap is also provided herein for attaching the helix of a human ear to medical (e.g., oxygen) tubing.

### BACKGROUND OF INVENTION

Beauticians frequently utilize hot implements such as curling irons, hot curlers, and straightening combs that are by design, very hot to the touch for processing the hair and occasionally such irons will cause uncomfortable heat or touch the ear causing pain. Although beneficial when used to curl hair, the irons, curlers and combs can be detrimental if brought into contact with the user's ear. Likewise, blow dryers can cause harm to sensitive ears due to the high intensity heat and noise from the blow dryer. Many women have experienced the pain that results from accidentally touching their ear with the iron. One slight touch is all that is required to receive a painful burn. In addition to the associated pain, the burn often leaves a noticeable burn mark or scab on the user. With the medical field, the ear may be irritated by oxygen equipment and other items rubbing on the ear.

There are numerous ear protecting devices. Roos, U.S. Pat. No. 2,148,838, discloses a means for protecting ears with celluloid, rubber or other materials that do not conduct heat and completely surrounds the ear thus making the protector warm and uncomfortable; Bogart, U.S. Pat. No. 2,763,869, also discloses a protector for ears using soft, flexible waterproof materials that requires an elastic cord to maintain the shower cap type design in position on the ear; Motley, U.S. Pat. No. 2,396,113, depicts uses what it calls a v-shaped recess construction made from plastics, asbestos or other suitable material with a large cut out to prevent interference with hearing; Barrington, U.S. Pat. No. 1,697,102, uses a curved portion that rests upon the outer portion of the ear similar to curved cover over the top of the ear with the same general shape on the outer and inner sides; and Suggs, U.S. Pat. No. 2,727,245, discloses a soft collapsible material fitting over the ear with a narrow wall clipping over the outer portion of the ear to fit tightly. However, these devices do not necessarily address all of the concerns necessary to protect an individual's ears during hair treating processes.

Other Ear protectors have been described in the patent literature. For example, U.S. Pat. No. 4,134,153 issued to Voorhees, U.S. Pat. No. 4,872,219 issued to Duncan, U.S. Pat. No. 5,257,420 issued to Byrne, Jr., U.S. Pat. No. 2,428,897 issued to Ungemah. A number of other attempts have been made to provide protecting devices. U.S. Pat. No. 7,614,089; U.S. Pat. No. 7,028,343; U.S. Pat. No. 6,298,493; U.S. Pat. No. 6,041,440; U.S. Pat. No. 5,920,912; U.S. Pat. No.5,816,254; U.S. Pat. No.4,935,965; U.S. Pat. No. 4,916,758; U.S. Pat. No. 4,308,623; and U.S. Pat. No. 3,452,365. These all describe ear covers or protectors, but fail to disclose an ear protector that is a flexible, comfortable material and with a shape that effectively protects against heat, noise and chemicals that is easy to install on the ear and remove.

### BRIEF DESCRIPTION OF THE DRAWINGS

These and other features, aspects, and advantages of the invention will become better understood with regard to the following description, appended claims, and accompanying drawings where:

FIG. 1 is a side view depicting a portion of the anatomy of a human ear;

FIG. 2 is side view of an outer side of one embodiment of an ear protector for a right ear;

FIG. 3 is front view of another embodiment of an ear protector for a right ear;

FIG. 4 is side view of an inner side of yet another embodiment of an ear protector for a right ear;

FIG. 5 is rear view of the embodiment of FIG. 2;

FIG. 6 is bottom view of the embodiment of FIG. 2;

FIG. 7 is sectional view along line 7-7 of FIG. 4 depicting a notch and membrane of the embodiment of FIG. 2;

FIG. 8 is perspective view of an inner side of the embodiment of FIG. 2;

FIG. 9 is another perspective view of an inner side of the embodiment of FIG. 2;

FIG. 10 is side view of an outer side of one embodiment of an ear protector for a left ear;

FIG. 11 is front view of the embodiment of FIG. 10;

FIG. 12 is side view of an inner side of the embodiment of FIG. 10;

FIG. 13 is rear view of the embodiment of FIG. 10;

FIG. 14 is bottom view of the embodiment of FIG. 10;

FIG. 15 is perspective view of an inner side of the embodiment of FIG. 10;

FIG. 16 is another perspective view of an inner side of the embodiment of FIG. 10;

FIG. 17 is side view of an outer side of another embodiment of an ear protector for a right ear;

FIG. 18 is front view of the embodiment of FIG. 17;

FIG. 19 is side view of an inner side of another embodiment of the embodiment of FIG. 17;

FIG. 20 is rear view of the embodiment of FIG. 17;

FIG. 21 is bottom view of the embodiment of FIG. 17;

FIG. 22 is sectional view along line 22-22 of FIG. 19 depicting a notch and membrane of the embodiment of FIG. 17;

FIG. 23 is perspective view of an inner side of the embodiment of FIG. 17;

FIG. 24 is another perspective view of an inner side of the embodiment of FIG. 17;

FIG. 25A is a perspective view of another ear protector embodiment with equipment tabs;

FIG. 25B is a top view of the embodiment of FIG. 25A;

FIG. 25C is a front view of the embodiment of FIG. 25A;

FIG. 26A is perspective view of yet another ear protector embodiment with equipment tabs;

FIG. 26B is a front view of the embodiment of FIG. 26A;

FIG. 27A is a perspective view of an ear and ear protector wrap;

FIG. 27B is another perspective view of the ear and ear protector wrap of FIG. 27A;

FIG. 28 is a perspective view of an ear protector wrap;

FIG. 29 is side view of the ear protector wrap of FIG. 28;

FIG. 30 is top view of the ear protector wrap of FIG. 28; and

FIG. 31 is bottom view of the ear protector wrap of FIG. 28.

### SUMMARY

In some embodiments, the present disclosure provides an ear protector including an inner wall, an outer wall, and a curved joining wall an upper helix cover, a middle tragus cover, and a lower lobule cover; a front portion, middle portion, and a rear portion; ear cavity button; and a notch; all comprising a thermoplastic elastomer material. The ear protector upper helix cover includes the inner wall connected with the curved joining wall connected with the outer wall, the middle tragus cover includes only the outer wall, and the lower lobule cover includes the outer wall, connected with the curved joining wall connected with the inner wall. The notch includes a membrane disposed in the upper helix cover, such that the membrane extends through the inner wall, the curved joining wall, and the outer wall. The membrane comprises a thinner thickness of the thermoplastic elastomer material, such that the notch extending front to rear is wide enough to allow the membrane to stretch and retract such that the ear protector opens and retracts allowing for opening the ear protector to extend around an ear and retract to remain on the ear in position as placed on the ear. The notch further includes an inner bridge and an outer bridge disposed in the center of the membrane, and further disposed on the interior of the upper helix cover, wherein the inner bridge is further disposed approximately between the inner wall and curved joining wall, and the outer bridge is disposed approximately between the outer wall and the curved joining wall, and the inner and outer bridge comprise a larger thickness of the thermoplastic elastomer material than the membrane for strengthening along the membrane and for comfort. The ear protector further includes a taper or a cutout disposed approximately between the upper helix cover and the tragus cover. The taper includes more security of the ear protector with the ear while the cutout allows for a wider variety of shapes and sizes of ears. The taper is disposed approximately between the upper helix cover and the tragus cover such that the upper helix cover presses on the top of the helix, the inner wall of the upper helix cover presses on the inner top of the helix, the outer wall of the upper helix cover presses on the outer top of the helix, and the ear cavity button fits below the helix of the ear and the taper presses on the top front of the helix to hold the ear protector securely on the ear. The cutout includes an open area abutting the curved joining wall of the upper helix cover disposed approximately in an area of the outer wall where the upper helix cover ends and where the tragus cover begins such that the ear protector may be used with a variety of ear shapes and sizes. The tragus cover is disposed in the front, middle portion of the ear protector and the tragus cover is also disposed with the front lower portion of outer wall such that shape of the middle tragus cover pushes a tragus portion of the ear for preventing damage to ear and for muffling sounds. The ear cavity button comprises mound of material disposed on the outer wall beginning at a rear lower edge of the membrane on the outer wall and extending forward along the outer wall edge of upper rear portion of the tragus cover wherein the ear cavity button increases in width and height from rear to front such that the ear cavity button fits below the helix of the ear providing secure holding of ear protector on the ear. In addition, the lower lobule cover includes the outer wall extending downward from the upper helix cover, the curved joining wall extending downward with a taper from the upper helix cover, and the inner wall extending downward from the upper helix cover with a taper from the upper helix cover, and the rear portion of the lower lobule cover curves downward and forward from the upper helix cover comprising a C-shape for conforming to the ear and for manipulating the ear upward and inward and for protecting the ear. The ear protector may include ear protector tabs.

In other embodiments, the present disclosure provides an ear protector wrap for attaching medical tubing to a helix of a human ear. The ear protector wrap may include:
a) continuous wall, the continuous wall having a first terminal end, a second terminal end, and a middle curvature portion between the first terminal end and the second terminal end;
b) a first substantially circular cavity formed by said continuous wall, said first substantially circular cavity having a first diameter;
c) a second substantially circular cavity formed by said continuous wall, said second substantially circular cavity having a second diameter, said second substantially circular cavity separated from said first substantially circular cavity by said middle curvature portion;
d) a first opening leading into said first substantially circular cavity, said first opening located between said first terminal end and said middle curvature portion; and
e) a second opening leading into said second substantially circular cavity, said second opening located between said second terminal end and said middle curvature portion.

### DETAILED DESCRIPTION OF THE INVENTION

The present invention relates to ear protection for the human ear. More specifically, the present invention discloses an ear protector and ear protector wrap providing a means for attaching with the ear and providing protection from heat, chemicals, and noise, and positioning the ear for hair treatments as well as irritation from medical equipment such as oxygen tubing that comes in contact with the ear. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

Overview of the Ear Protector:

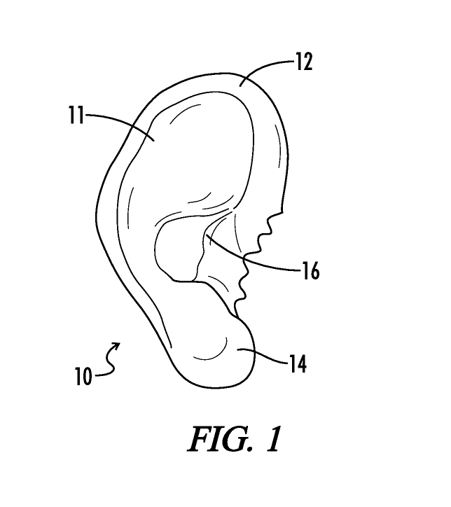

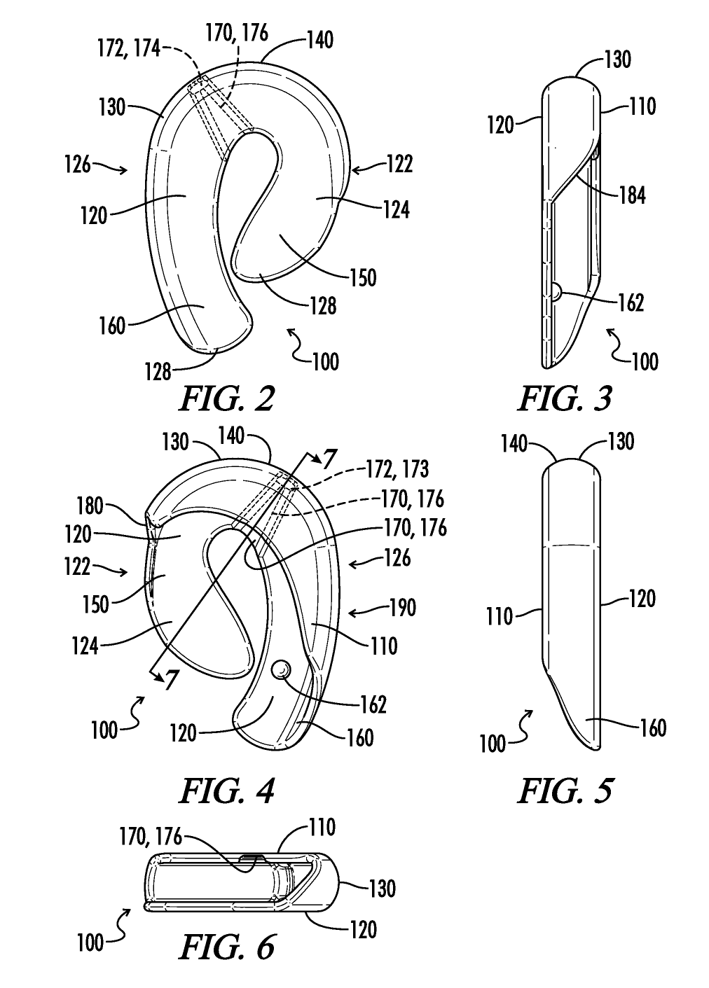

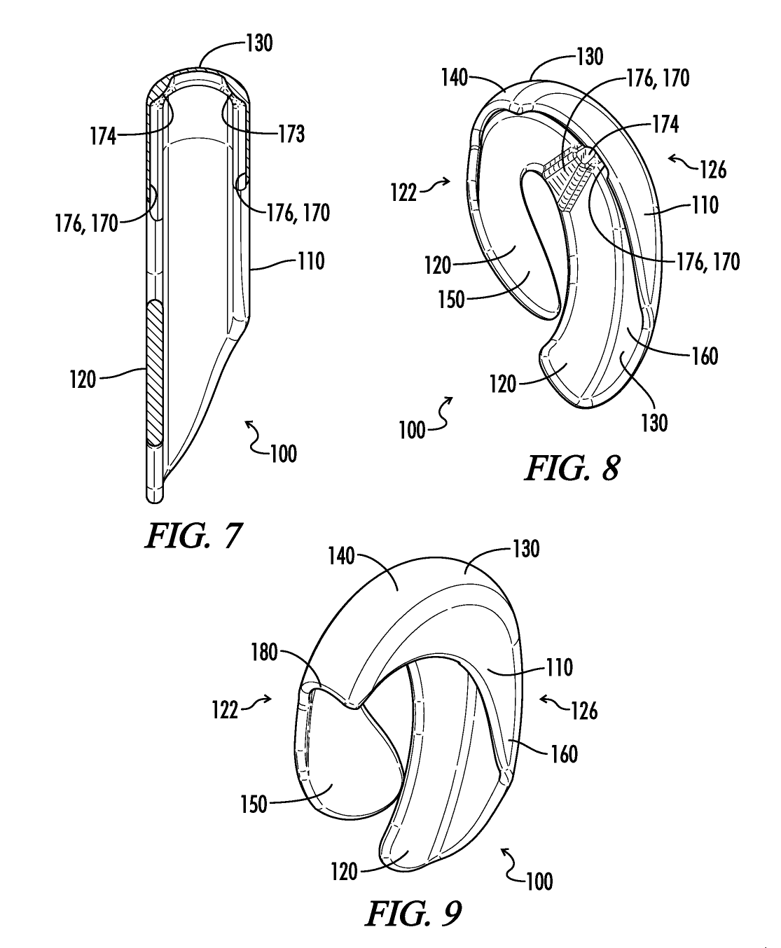

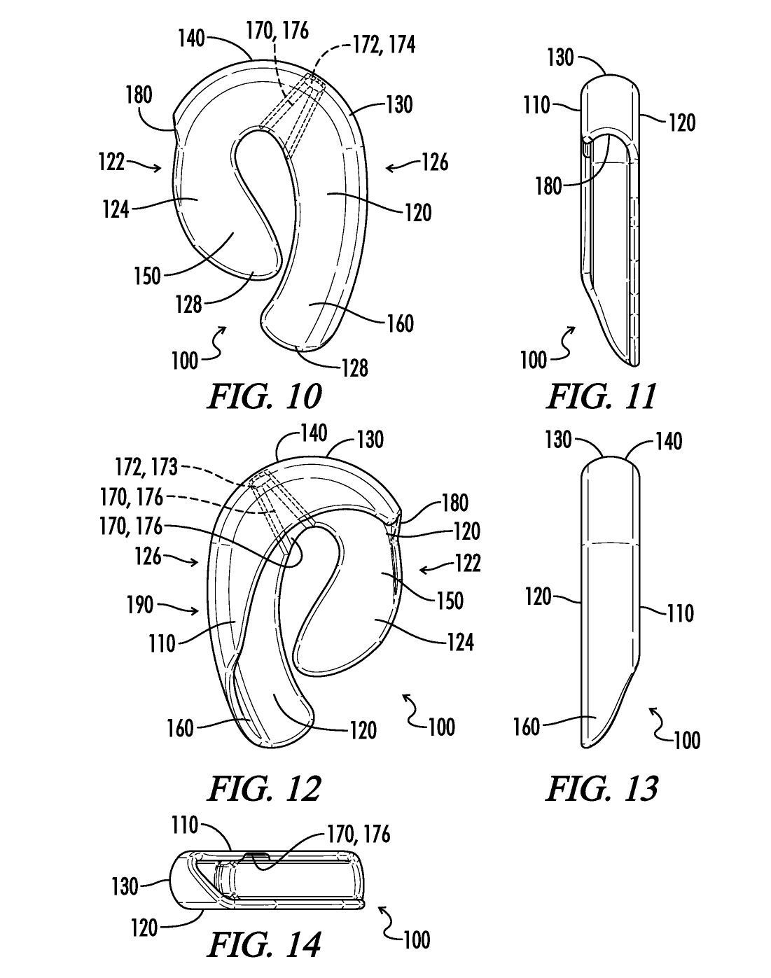

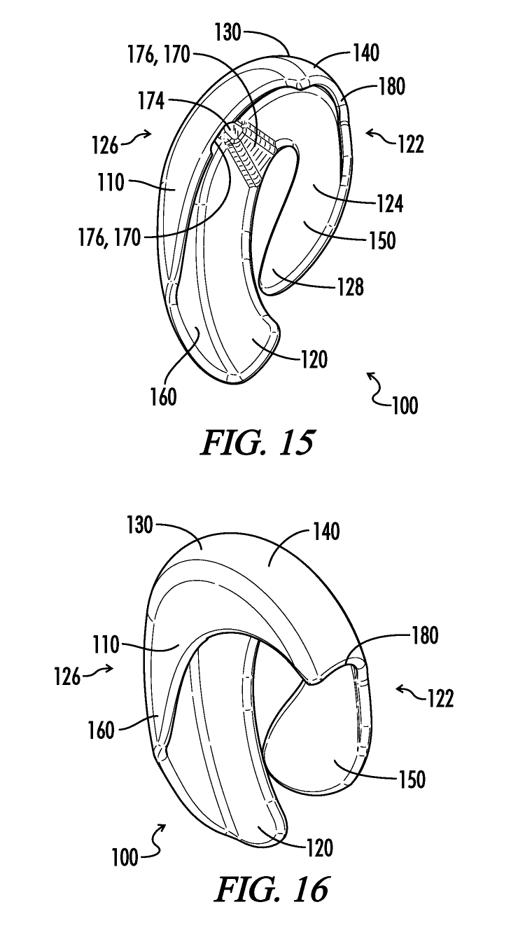

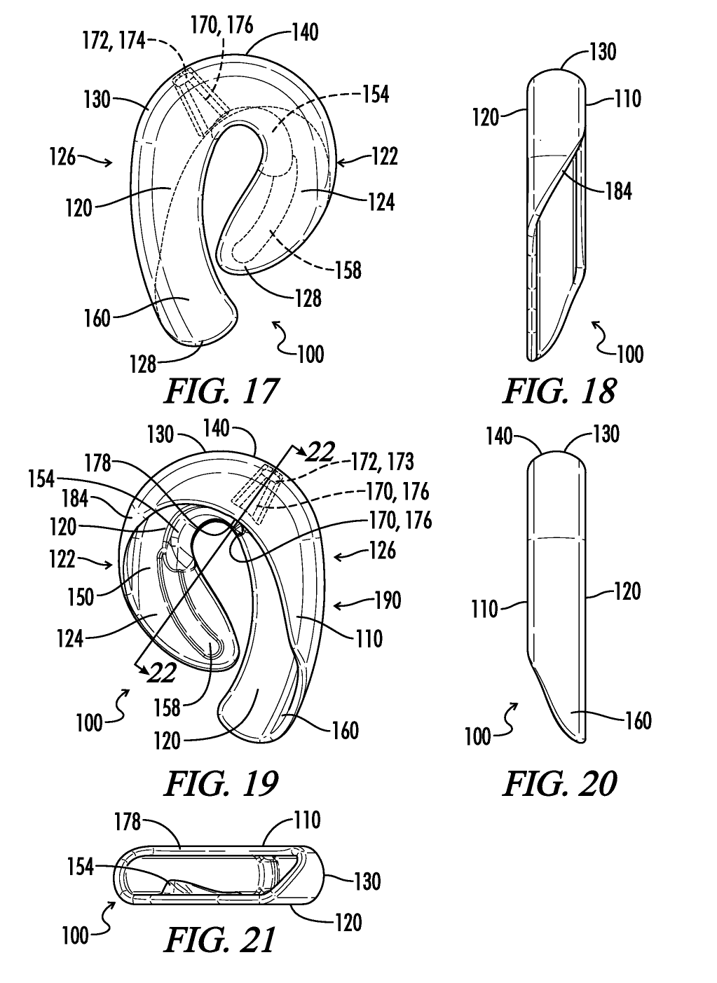

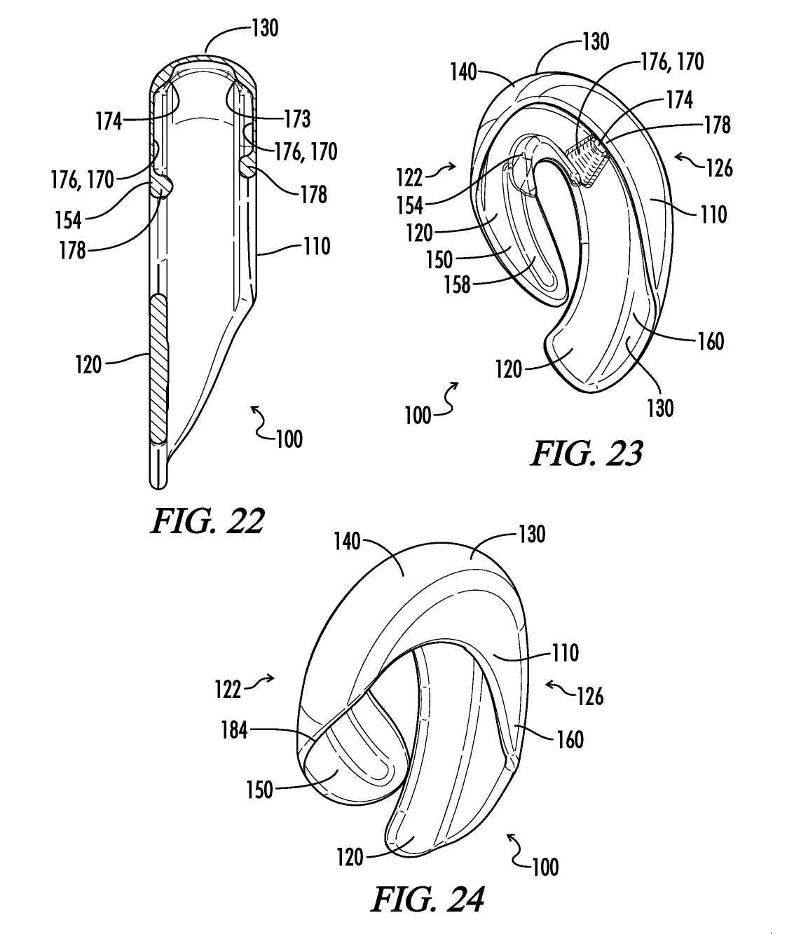

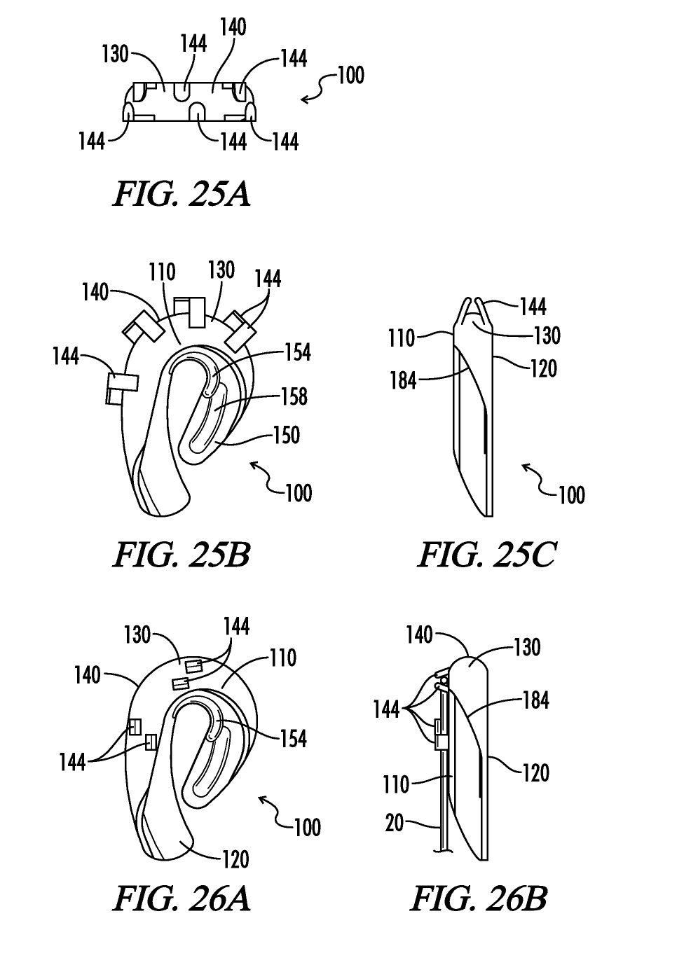

As depicted in FIGS. 1 through 26B, an ear protector 100 may provide a means for covering an ear 10, manipulating the ear 10, and protecting the ear 10 from rubbing, chemicals, and heat. FIGS. 2 through 9 are figures of different embodiments of the ear protector for the right ear; FIGS. 10 through 16 are figures of different embodiments of the ear protector for the left ear; FIGS. 17 through 24 are figures of embodiments of the ear protector for the right ear wherein the figures for the left ear would be the mirror view of the figures for the right ear; and FIGS. 25A through 26B are figures of embodiments of the ear protector for the right ear wherein the figures for the left ear would be the mirror view of the figures for the right ear. The ear protector 100 may comprise three walls: an inner wall 110, an outer wall 120, and a curved joining wall 130. The ear protector 100 may comprise covers for three areas of an ear: an upper helix cover 140, a middle tragus cover 150, and a lower lobule cover 160. The ear protector 100 may have three general areas: a front portion 122, middle portion 124, and a rear portion 126. In addition, the ear protector 100 is manufactured with a material this is flexible, able to resume its original shape when a deforming force is removed, light weight and low density, has a high compression set, has insulation properties and chemical resistance properties, is easy to manufacture, and is easily colored and processed. The ear protector 100 may further include a notch 170 with a thin membrane 176 for stretch and retraction, and thicker bridges 172, 173, 174, 178 for strength and comfort, all for ease of installing and removing ear protector 100 with the ear 10. In addition, an ear cavity button 154 may be provided to securely hold the ear protector 100 in place on the ear, and an end support bridge 178 may be added for additional strength where the notch membrane 176 joins an edge of the ear protector 100. The ear protector 100 may comprise equipment tabs 144 as depicted in FIGS. 25A through 25C on the top of the upper helix cover or as depicted in FIGS. 26A and 26B on the outside of the inner wall 110 of the ear protector 100 for securing and holding equipment such as oxygen tubing, hoses, or lines 20 that would normally wrap around the ear.

Design Specifications:

As illustrated in FIGS. 2 through 26B, an ear protector 100 structure may include an inner wall 110, an outer wall 120, and a curved joining wall 130 made of a thermoplastic elastomer material (TPE) and soft flexible elastic polyvinyl chloride (PVC). An upper helix cover 140, and a lower lobule cover 160 may comprise the inner wall 110, the outer wall 120, and the curved joining wall 130, while a middle tragus cover 150 may comprise the outer wall 120.

The upper helix cover:

As shown in FIGS. 1 through 26B, the upper helix cover 140 structure comprises the inner wall 110, connected with the curved joining wall 130, connected with the outer wall 120, and a notch 170. The structure of the notch 170 includes a membrane 176 disposed in the upper helix cover 140, such that the membrane 176 extends through the inner wall 110, the curved joining wall 130, and the outer wall 120. The membrane 176 comprises a thinner thickness of the thermoplastic elastomer material, wherein the notch 170 extends front to rear, wide enough and the thickness of the material thin enough, to allow the membrane 176 to stretch and retract such that the ear protector 100 opens and retracts allowing for opening the ear protector 100 to extend around an ear 10 and retract to remain on the ear 10 in position as placed on the ear 10. The notch 170 further includes a support bridge 172 that may include an inner support bridge 173 and an outer support bridge 174 disposed in the center of the membrane 176, disposed on interior of the upper helix cover 140, such that the inner support bridge 173 is further disposed approximately where the inner wall 110 transitions to the curved joining wall 130, and the outer support bridge 174 is disposed approximately where the outer wall 120 transitions to the curved joining wall 130, wherein the inner support bridge 173 and the outer support bridge 174 comprise a larger thickness of the thermoplastic elastomer material than the membrane 176 for strengthen of the ear protector 100 along the membrane 176 and for comfort as the upper helix cover 140 rests on the helix 12 of the ear 10. In addition, end support bridges 178 may be added for additional membrane 176 strength where the membrane 176 joins the edges of the ear protector 100.

The middle tragus cover:

The middle tragus cover 150 structure includes primarily the outer wall 120. A portion of the curved joining wall 130 may adjoin the tragus cover 150. The tragus cover 150 is disposed on the front 122, middle portion 124 of the ear protector 100 and on the front 122 lower portion 128 of the outer wall 120 such that shape of the tragus cover 150 pushes or covers the tragus portion 16 of the ear 10 preventing ear damage and muffling sounds. The middle tragus cover 150 structure may further comprise an ear cavity button 154. The ear cavity button 154 is a narrow and lower mound of material beginning at the rear edge of the membrane 178 outer wall 120 extending forward around the edge of the ear protector areas of the middle tragus cover 150 and the lower lobule cover 160 with the ear cavity button 154 mound of material increasing in width and height in the forward position and decreasing in width and height in the rear position. The ear cavity button 154 follows the edge of the ear protector area of the middle tragus cover 150 and the lower lobule cover 160. The ear cavity button 154 fits below the helix of the ear such that the ear cavity button 154 presses on the helix of the ear while the upper helix cover 140 presses on the top of the helix, the inner wall 110 of the upper helix cover 140 presses on the inner top portion of the ear helix, the outer wall of the upper helix cover 140 presses on the outer top of the ear helix, and the C-shape of the lower lobule cover presses on the rear of the ear lobule providing snug but comfortable holding of the ear protector 100 securely in place such that medical equipment and lines such as oxygen hoses may be attached with the ear protector without irritating the ear 10. The tragus cover may further comprise tragus cover ribs 158 comprising thicker material disposed from said ear cavity button extending downward toward bottom of said tragus cover such that said tragus cover rib 158 of said middle tragus cover pushes a tragus portion of said ear for preventing damage to ear and for muffling sounds.

The lower lobule cover:

The structure of the lower lobule cover 160 includes the outer wall 120, the curved joining wall 130 and the inner wall 110. The lower lobule cover 160 includes the outer wall 120 extending downward from the upper helix cover 140, the curved joining wall 130 extending downward with a taper from the upper helix cover 140, and the inner wall 110 extending downward with a taper from the upper helix cover 140. The rear portion 126 of the lower lobule cover 160 curves downward and forward from the upper helix cover 140 comprising a C-shape 190 for conforming to the ear 10 and for manipulating the ear 10 upward and inward and for protecting the lobule 14 of the ear 10. As shown in FIGS. 3 and 4, the lower lobule cover 160 may comprise a lower lobule cover bump 162 comprising a mound of material thicker than the outer wall 120 of the lower lobule cover 160 disposed on the outer wall 120 of the lower lobule cover 160 to aid in securing the ear protector 100 in place on the ear 10.

The cutout or taper:

The ear protector 100 may comprise an upper helix cover cutout 180 disposed approximately between the upper helix cover 140 and the tragus cover 150, wherein the upper helix cover cutout 180 includes an open area, an area without material, on the curved joining wall 130 of the upper helix cover 140. The upper helix cover cutout 180 is disposed in an area of the outer wall 120 approximately where the upper helix cover ends 140 and where the tragus cover 150 begins such that the ear protector 100 may be used with a variety of shapes and sizes of ears 10. The upper helix cover cutout 180 disposition may be adjusted upward or downward along the curved joining wall 130 for an even wider variety of shapes and sizes of ear 10. As illustrated in FIGS. 2, 3, 17 through 19, 21, 23 through 26B instead of the upper helix cover cutout 180, a taper or protective angle 184 may be included on the curved joining wall 130 wherein the taper 184 may begin where the inner wall 110 transitions to the curved joining wall 130, and may extends downward across the curved joining wall 130 toward a point approximately midway 124 down the front portion 122 of the tragus cover 150 where the curved joining wall 130 may transition to the outer wall 120. The protective angle or taper 184 provides more protection of the ear 10 and better holding of the ear protector 100 in relation with the ear. When the taper 184 is used in combination with the ear cavity button 154, the ear protector 100 is held very securely on the ear 10.

The equipment tabs:

The ear protector 100 may comprise equipment tabs 144 as depicted in FIGS. 25A through 25C on the top of the upper helix cover or FIGS. 26A and 26B for securing and holding equipment such as oxygen hoses or lines 20 that would normally wrap around the ear. The equipment tabs 144 may comprise pairs of tabs 144 with elasticity and are angled such that equipment or tubing may be inserted between the tabs 144 and the tabs 144 receive the equipment and retain it in position on the ear protector 100 until the equipment is pulled away from the tabs 144. One embodiment is show with the equipment tabs 144 disposed on the upper helix cover 140 and another embodiment depicts the equipment tabs 144 on the inner wall 110 of the ear protector 100.

The ear protector wrap 200:

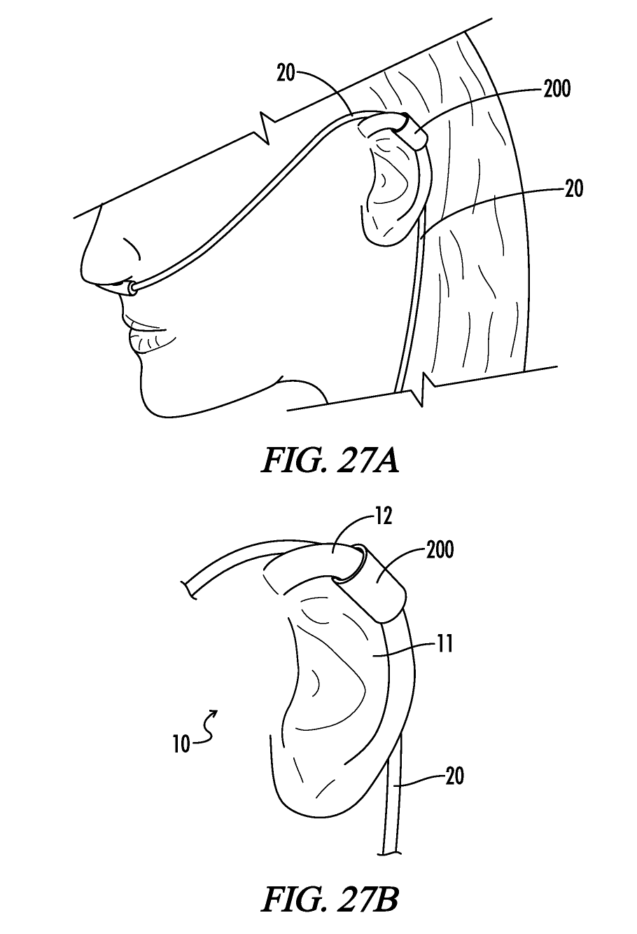

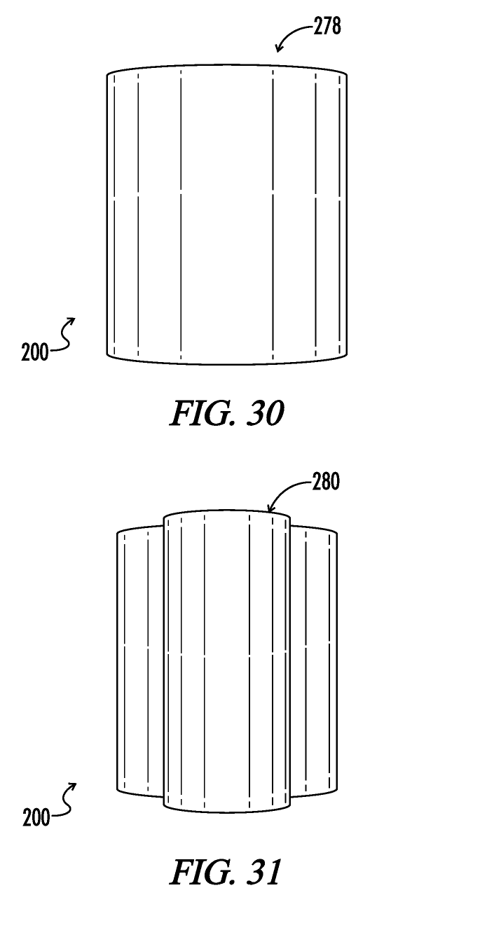

The present disclosure also provides an ear protector wrap 200 for attaching medical tubing 20 (e.g., oxygen tubing) to the helix 12 of a human ear 10. Preferably, the ear protector wrap 200 is used in place of the ear protector 100 described above and shown in FIGS. 1-26. As shown in FIGS. 27-31, the ear protector wrap 200 includes a continuous wall 220, which has a first terminal end 268, a second terminal end 228, and a middle curvature portion 240 between the first terminal end and the second terminal end. The continuous wall 220 may be a single piece (e.g. of plastic), as shown in FIGS. 27-31, or alternatively, it may be comprised of multiples pieces that form a continuous wall. The ear protector wrap 200 further includes a first substantially circular cavity 280 formed by said continuous wall curving upon itself, said first substantially circular cavity having a first diameter 276; a second substantially circular cavity 278 formed by said continuous wall curving upon itself, said second substantially circular cavity having a second diameter 236, said second substantially circular cavity separated from said first substantially circular cavity by said middle curvature portion; a first opening 270 leading into said first substantially circular cavity, said first opening located between said first terminal end and said middle curvature portion; and a second opening 230 leading into said second substantially circular cavity, said second opening located between said second terminal end and said middle curvature portion. The first diameter 276 may be of equal size, longer or shorter than the second diameter 236, depending on the size of the medical tubing 20 and the helix 12 of the human's ear 10. The ear protector wrap 200 may be interchangeable for the right or left ear. The same embodiment of an ear protector wrap 200 may be rotated such that the longer diameter may be attached with the helix 12 of the human's ear 10 or with the medical tubing 20.

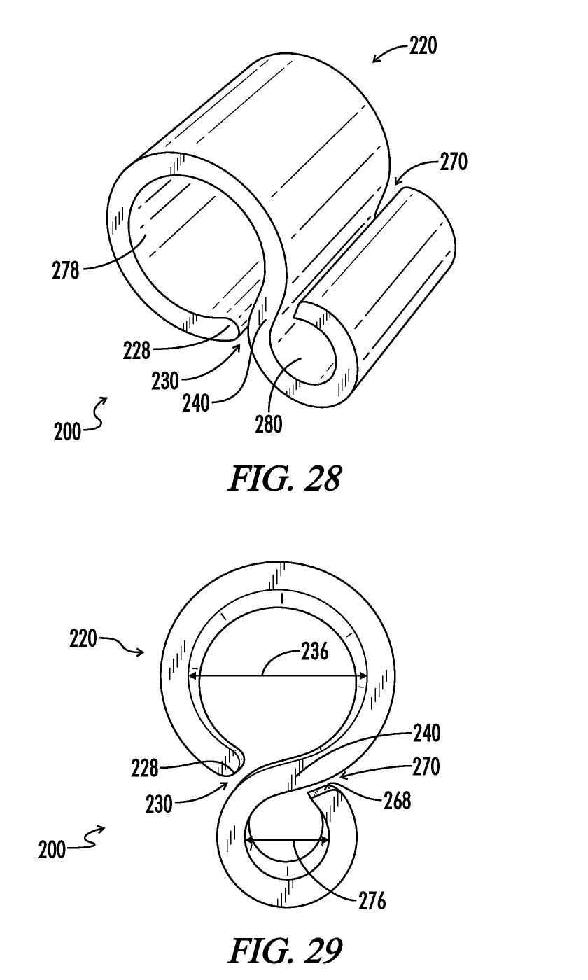

Optionally, the ear protector wrap 200 is in the form of a truncated FIG. 8, as shown in FIGS. 28-31. Preferably, said first opening 270 is configured to allow said medical tubing 20 to pass through said first opening and into said first substantially circular cavity 280. Preferably, said second opening 230 is configured to allow said helix 12 of said human ear 10 to pass through said second opening and into said second substantially circular cavity 278. Optionally, the ear protector wrap 200 consists essentially of the components described above.

In some embodiments, the ear protector wrap 200 is used in a method for attaching medical tubing 20 to a helix 12 of a human ear 10, the method comprising:
a) providing the ear protector wrap 200;
b) passing said medical tubing 12 through said first opening 270 and into said first substantially circular cavity 280; and
c) passing said helix 12 of said human ear 10 through said second opening 230 and into said second substantially circular cavity 278.

Steps b) and c) may be performed in any suitable order, including simultaneously, and the method may include additional steps.

Optionally, after steps a) and b) said first terminal end 268 or said second terminal end 270 may contact the scapha 11 of the ear 10. (The scapha 11 is the groove between the helix 12 and anti-helix of the ear 10).

The material:

The preferred material for the ear protector 100 and ear protector wrap 200 is a plastic, such as thermoplastic elastomer (TPE) and/or soft flexible elastic polyvinyl chloride (PVC). There are a number of TPE or PVC products currently on the market. The preferred embodiment for the ear protector and wrap material TPE and PVC is SOFT TOUCH (Spartech Plastics, Clayton, Mo.). However, SARLINK (Teknor Apex, Pawtucket, R.I.), HYTREL (Dupont), and SANTOPRENE (Exxon Mobil) materials may also be used for the ear protector and ear protector wrap. However, other materials with the same characteristics may be used and new products with similar characteristics may be used as they become available.

Characteristics and reasons for using thermoplastic elastomer materials such as SOFT TOUCH, SARLINK, HYTREL, and SANTOPRENE:
A) User Friendly: The material is easy to handle and easy to hold on to.
B) Flexible: The material makes the “hinge” that allows the ear guard to hold onto, and stay on the ear while in use, giving confidence to the user.
C) Elastomer: The material allows the ear protector to perform like a natural rubber part and remain flexible, so it doesn't harm the user while worn. This characteristic gives the product a natural fit and feel. The elastomeric properties is useful to the “hinge” design by allowing the ear protector to expand, and then recover so the part slips over the ear, and then also return with enough grip to remain functional and to remain in position on the ear.
D) Low weight: This characteristic of the material provides comfort for the user.
E) High compression set: This characteristic of the material keeps the product useable while in storage thus adding to the long life of the product.
F) Insulation properties: This characteristic of the material prevents heat from any of the heat sources from being transmitted directly to the ear. The wearer of the product feels enough heat to know that heat sources are close to the ear, but the ear protector helps prevent burning the ear.
G) Chemical resistance: This characteristic of the material insures the product remains functional even while exposed to a variety of chemicals that can come in contact with the ear protector thru contact with the hair.
H) Ease of manufacturing: SOFT TOUCH, SARLINK, HYTREL, and SANTOPRENE materials are easily colored, and processed on standard thermoplastic equipment.

Advantages and features of the ear protector and the ear protector wrap:
1. The heat resistant ear protector covers, pushes down, or manipulates the ear away from the head to make it easier to cut the hair or apply chemicals. The skin in this area is thin and very sensitive wherein the covers can protect the skin and ear from heated items such as flat irons, curling irons, and hot rollers. The ear protector also may be comfortably worn during hair treatments to protect ears from any liquids or semi-liquids required by such treatments. Some of the chemicals used with hair styling may cause acute discomfort. In addition, the ear protector covers protect the ears from medical equipment such as oxygen hoses rubbing and irritating the ears.
2. The ear protector has a notch all the way across the width of the ear protector through a cross-section of the upper helix cover. The notch area is thinner with a thin membrane that allows for the opening of the ear protector in order to place the ear protector on the ear. When released from the open position, the ear protector springs back around the ear to keep the ear protector in place. In the center of the membrane, approximately at the top of the membrane on each side of the upper helix cover, thicker material is placed for added comfort as well as to strengthen the ear protector along the membrane. The thicker material associated with the notch and the membrane is located along the inside, or ear side, of the ear protector where the thicker material forms a bridge on the inside of each side of the upper helix cover and the membrane edge of the ear protector. To further aid in securing the ear protector with the ear an ear cavity button may be included. The ear cavity button in combination with the taper provides snug, comfortable securing of the ear protector.
3. The ear protector outer shape from the upper portion of the ear protector moving rearward moves down and curves around the ear so the lobule of the ear is covered, protecting the lobule of the ear from heat and rubbing while also helping to pull the ear up or manipulating the lower half of the ear that is covered when cutting the hair.
4. The tragus cover is shaped to prevent damage to the ear. The tragus cover also helps to push in the tragus which helps muffle sounds while cutting the hair around the ears using hair clippers.
5. The ear protector in front of the upper helix cover may have a cutout to allow for effective use with a variety of ear shapes and sizes. However, where protection of the ear and security or holding of the ear protector is more of a concern, the taper instead of the cutout provides for more security and ear protection.
6. The ear protector is also wrapped around the back of the ear thus pulling the ear inward, making a C-clamp shape.
7. The ear protector may include tabs along the top of the upper helix cover for holding medical equipment such as an oxygen hose. This allows the ear protector to prevent irritation and rubbing of the sensitive portion of the ear while patients need to utilize oxygen through tubing that would normally wrap around the ear wherein movement rubs and irritates the ear.
8. The ear protector wrap provides a means of attaching medical equipment such as nasal cannula tubing that comes in contact with the ear on the ear. Nasal cannula (oxygen tubing) causes decubitus ulcers, pressure sores back of ear pain correlated with oxygen use. The ear protector wrap allows medical equipment such as nasal cannula to directly bypass the back of the ears and will also keep tubing from being so tight against the face where sores can also occur. Ear protector wraps keep these areas looser so friction doesn't occur that causes sores, also more than one ear wrap can be used on each ear. Ear protector wraps may include a substantially circular cavity for nasal cannula, to be pressed in around medical equipment to secure along back side of ear wrap that helps support medical equipment away from the back of ear. The first terminal end of the ear protector wrap fits snug into the scapha of the human ear.

What is claimed is:

1. An ear protector wrap for attaching medical tubing to a helix of a human ear, the wrap comprising:
a) continuous wall, the continuous wall having a first terminal end, a second terminal end, and a middle curvature portion between the first terminal end and the second terminal end;
b) a first substantially circular cavity formed by said continuous wall curving upon itself, said first substantially circular cavity having a first diameter;
c) a second substantially circular cavity formed by said continuous wall curving upon itself, said second substantially circular cavity having a second diameter, said second substantially circular cavity separated from said first substantially circular cavity by said middle curvature portion;
d) a first opening leading into said first substantially circular cavity, said first opening located between said first terminal end and said middle curvature portion; and
e) a second opening leading into said second substantially circular cavity, said second opening located between said second terminal end and said middle curvature portion.

2. The ear protector wrap of claim 1, wherein said first diameter is larger than said second diameter.

3. The ear protector wrap of claim 1, wherein said ear protector wrap is in the form of a truncated FIG. 8.

4. The ear protector wrap of claim 1, wherein said first opening is configured to allow said helix of said human ear to pass through said first opening and into said first substantially circular cavity.

5. The ear protector wrap of claim 1, wherein said second opening is configured to allow said medical tubing to pass through said second opening and into said second substantially circular cavity.

6. The ear protector wrap of claim 1, wherein said first opening is configured to allow said medical tubing to pass to pass through said first opening and into said first substantially circular cavity.

7. The ear protector wrap of claim 1, wherein said second opening is configured to allow said helix of said human ear through said second opening and into said second substantially circular cavity.

8. The ear protector wrap of claim 1, wherein said continuous wall is comprised of plastic.

9. The ear protector wrap of claim 8, wherein said continuous wall is comprised of thermoplastic elastomer.

10. The ear protector wrap of claim 8, wherein said continuous wall is comprised of polyvinyl chloride.

11. A method for attaching medical tubing to a helix of a human ear, the method comprising:
a) providing the ear protector wrap of claim 1;
b) passing said helix of said human ear through said first opening and into said first substantially circular cavity; and
c) passing said medical tubing through said second opening and into said second substantially circular cavity.

12. The method of claim 11, wherein said human ear further comprises a scapha and wherein after steps a) and b) said first terminal end contacts said scahpa.

13. An ear protector wrap for attaching medical tubing to a helix of a human ear, the wrap consisting essentially of:
a) continuous wall, the continuous wall having a first terminal end, a second terminal end, and a middle curvature portion between the first terminal end and the second terminal end;
b) a first substantially circular cavity formed by said continuous wall curving upon itself, said first substantially circular cavity having a first diameter;
c) a second substantially circular cavity formed by said continuous wall curving upon itself, said second substantially circular cavity having a second diameter, said second substantially circular cavity separated from said first substantially circular cavity by said middle curvature portion;
d) a first opening leading into said first substantially circular cavity, said first opening located between said first terminal end and said middle curvature portion; and
e) a second opening leading into said second substantially circular cavity, said second opening located between said second terminal end and said middle curvature portion.
